(19) United States
(12) Patent Application Publication
(10) Pub. No.: US 2022/0111036 A1
(43) Pub. Date: Apr. 14, 2022

(54) METHOD FOR DEFINING A PERSONALIZED VACCINE AGAINST HIV/AIDS

(71) Applicants: Ricardo Diaz, Barueri (BR); Andrea Savarino, Vinovo (IT); Iart Shytaj, São Paulo (BR)

(72) Inventors: Ricardo Diaz, Barueri (BR); Andrea Savarino, Vinovo (IT); Iart Shytaj, São Paulo (BR)

(73) Assignee: Daniele Cunha, São Paulo (BR)

(21) Appl. No.: 17/547,350

(22) Filed: Dec. 10, 2021

Related U.S. Application Data
(63) Continuation of application No. PCT/BR2020/050204, filed on Jun. 10, 2020.
(60) Provisional application No. 62/859,286, filed on Jun. 10, 2019.

(51) Int. Cl. A61K 39/21; A61K 31/455; A61K 31/7135; A61P 31/18

(52) U.S. Cl. CPC A61K 39/21; A61K 31/455; A61K 2039/5158; A61P 31/18; A61K 31/7135

(57) ABSTRACT

A novel approach to the development of a personalized vaccine. This approach is based on: A) sequencing of the gag gene from an HIV-infected individual treated with antiretroviral therapy; B) sequencing of the HLA alleles of the same individual; C) selecting the epitopes recognized by the individual's own HLA Class I within the highly-conserved Gag₂₅₆₋₃₇₇, Gag₁₄₇₋₁₆₉ and/or Gag₂₂₅₋₂₅₁ amino acid sequences. An original algorithm that designs the target peptide for the vaccine starting from viral and HLA sequences of an individual with HIV/AIDS, forms the core of the present invention. The original algorithm makes extensive use of existing open- source software for protein design. The peptides designed in this manner and accordingly synthesized may be exploited as a therapeutic vaccine against HIV/AIDS. Vehicles for such peptides may be an individual's own dendritic cells pulsed with the peptide combination or a specific viral or DNA vector leading to intracellular expression of the viral peptides. The present vaccine approach may contribute to control of viremia once antiretroviral therapies are suspended.

This application is a continuation of International Appl. No. PCT/BR2020/050204, filed Jun. 10, 2020, which claims the benefit of U.S. Provisional Patent Appl. Ser. No. 62/859,286, filed Jun. 10, 2019, both of which are incorporated herein by reference.

### INTRODUCTION

This report refers to a patent application for an invention of a novel approach to the development of a personalized vaccine. To summarize, this approach is based on: A) sequencing of the gag gene from an HIV-infected individual treated with antiretroviral therapy; B) sequencing of the HLA (acronym for human leukocyte antigen) alleles of the same individual; C) selecting the epitopes recognized by the individual's own HLA Class I within the highly-conserved Gag₂₅₆₋₃₇₇, Gag₁₄₇₋₁₆₉ and/or Gag₂₂₅₋₂₅₁ amino acid sequences.

### STATE OF THE ART

It is known that finding a vaccine for HIV/AIDS has been a Holy Grail in biomedical research for decades. So far, the efforts of the scientific community have been frustrated by the complexity of HIV biology and the virus' ability to mutate and escape the host's immune response. In the search for cure or treatment, two types of vaccines have been postulated, preventive and therapeutic. The first impedes the establishment of infection before exposure of the organism to the virus, while the latter enables the patient to elicit immune responses to keep the virus in check once infection has been established. For both purposes, several viral antigens or combinations have been proposed with mixed or disappointing results [Ensoli et al., Retrovirology 2016 (https.retrovirology.biomedcentral.com/track/pdf/10.1186.s12977-016-0261-1.pdf) ; Lelièvreet al., IAS 2017 (9th IAS Conference on HIV Science 23-26 July 2017); Angel et al., AIDS 2011

(https://joumals.lww.com/aidsonline/fulltext/2011/03270/A randomized contr olled trial of HIV therapeutic.2.aspx) ; Gay et al., AIDS Res Hum Retrovirus 2017; Picker et al., CROI 2017 (http://www.croiwebcasts.org/console/player/33440); Burton et al., PNAS 2015 (https://www.pnas.org/content/early/2015/08/05/1513050112); Vandekerckhove et al., EACS 2017 (https://resourcelibrary.eacs.cyim.com/) 1.

Although different antigens have been postulated to form the basis of preventive or therapeutic vaccination against HIV (neutralizing antibodies against HIV surface glycoproteins inhibit viral infectivity in the case of preventive vaccines, and cytotoxic cell-mediated immune responses targeting intracellular viral antigens in the case of therapeutic vaccines), there is, at present, no evidence in favor of any of the options except evidence that excludes the hypothesis that a single vaccine approach may act both as a preventive and as a therapeutic vaccine.

Cell-mediated immunity against the viral capsid gag protein represents one of the few immunological correlates of protection against disease progression. HIV-infected individuals in whom the disease progresses slowly (long-term non-progressors) or who do not develop AIDS due to very low/undetectable virus levels in blood (elite controllers) often show evidence of active anti-Gag cell mediated responses [Addo et al., J Virol 2003 (https://pubmed.ncbi.nlm.nih.gov/12525643/); Edwards et al. J Virol 2002 (https://pubmed.ncbi.nlm.nih.gov/11836408/) Frahm et al., J Virol 2004 (https://pubmed.ncbi.nlm.nih.gov/pmc/articles/PMC369231); Hunt et al. J Infect Dis 2008; (https://academic.oup.com./jid/article/197/1/126/796374?login=true); Julg et al. J Virol 2010 (https://www.ncbi.nlm.nih.gov/pmc/articles/PMC2876607/); Kiepiela et al. Nat Med 2007 (https://pubmed.ncbi.nlm.nih.gov/17173051/); Stephenson et al. J Virol 2012 (https://jvi.asm.org/content/jvi/86/18/9583.full.pdf); Ziniga et al. J Virol 2006 (https://pubmed.ncbi.nlm.nih.gov/16501126/)]. Unlike the immune responses directed against other viral antigens, the anti-gag responses are associated with a lower viral set point, i.e. the level of virus that remains stable in blood plasma during the prolonged steady state of the disease [ibidem].

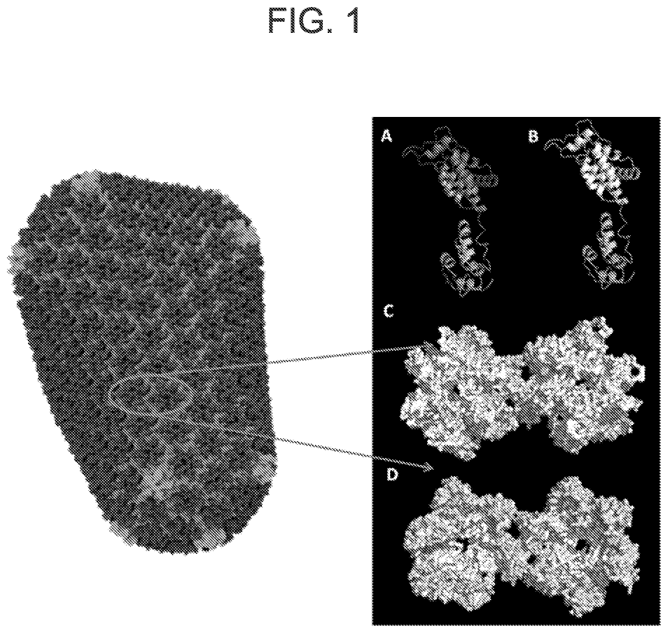

Gag is a pivotally important immunogen as it is fundamental in determining the virus internal structure. In particular, one of its maturation products, (p24), is the building block of the viral capsid, the icosahedral core of the virus protecting the two genomic RNA molecules (as illustrated in FIG. 1 of the attached Drawings). Different from the envelope glycoproteins, p24 is not exposed on the cell surface-derived lipoid vesicle that surrounds the viral capsid and is not free to float on the virus surface. Several constrains lock p24 within the icosahedral structure and limit its capability to mutate. However, immune-escape mutations of Gag have been described [Burwitz BJ et al. Retrovirology 2012 (htfps ://retrovirology.biomedcentral.com/articles/10.1186/1742-4690-9-91)1. Some of these can induce viral replication and thus revert the elite control condition.

The immune correlates of the post-treatment control of viremia that we observed in two macaques infected with the HIV homolog SIVmac251 have recently been described [Shytaj et al. J Virol 2015 (https://jvi.asm.org/content/89/157521)]. The macaques had received antiretroviral therapy (ART) in combination with an experimental treatment with immune modulating drugs.

Although the virus was not eradicated, the macaques showed, upon suspension of all therapies, a condition reminiscent of elite control (i.e., post-therapy control), which was associated with anti-Gag cell-mediated immunity [Shytaj et al. J Virol 2015]. The strongest immune responses were directed against an amino acid sequence highly conserved in both human and simian lentiviruses (Gag₂₅₆₋₃₆₇; amino acid numbering is according to the Gag epitope map in the Los Alamos HIV database: https://www.hiv.lanlgov/content/immunology/maps/ctl/Gag.html: accessed Jun. 8 2019) at the C-terminal region of SIVmac251 p27 (homologous to HIV-1 p24). When this sequence was mapped onto a three-dimensional structure of SIVmac251 p27, it was found to correspond to a portion of the protein responsible for the multimerization of p27 hexamers (p24, as well as p27 multimers have a recursive pattern with the hexamer being the fundamental unit and hexamers of hexamers being necessary for further assembly of the capsid structure) [Shytaj e Savarino J Med Primatol 2015 (https://pubmed.ncbi.nlm.nih.gov/26058990/)]. In these macaques, no escape mutations were observed, and they maintained long-term control of viremia [Shytaj et al., J Virol 2015].

Independently conducted analyses have also shown that the C-terminal protein portion of Gag, due to its high level of conservation, is an attractive vaccine target [Munson et al., Hum Vaccin Immunother 2018 (https://pubmed.ncbi.nlm.nih.gov/29648490/)]Immunization of macaques with selected peptides from the highly conserved regions of Gag was later shown to elicit significant immune responses, which are not stimulated under standard pathological conditions when the immune system is stimulated by multiple and highly immunogenic viral antigens.

Despite the evidence reviewed above, the problem of finding a vaccine against HIV/AIDS will not simply be solved by immunizing human individuals with manufacturer-standardized peptides, even when they are derived from highly conserved Gag regions. To elicit strong CD8⁺ T-lymphocyte responses, peptides should show optimal binding to an individual's own HLA Class I molecules, which are proteins specialized in presenting intracellular antigens to CD8⁺ T-cells and showing different specificities for the different portions of an antigen. Moreover, although the Gag₂₅₆₋₃₆₇ sequence is highly conserved, a certain degree of variation among different HIV-1 clades and strains is observed [Shytaj e Savarino, J Med Primatol 2015]. Therefore, it is possible to hypothesize that only a personalized medicine approach, taking into account both the virus' and the host's genetic variability may be able to efficiently elicit the best immune response.

However, the present invention shows that not all of the automated procedures for custom peptide design may become successful for the purpose of finding a cure for HIV/AIDS, because, as detailed below, only the algorithm herein disclosed for the first time eventually led to post-therapy control of HIV in patients. Finally, the right ‘conditioning regimen’ should be applied for a successful therapeutic vaccine.

### OBJECTIVES OF THE INVENTION

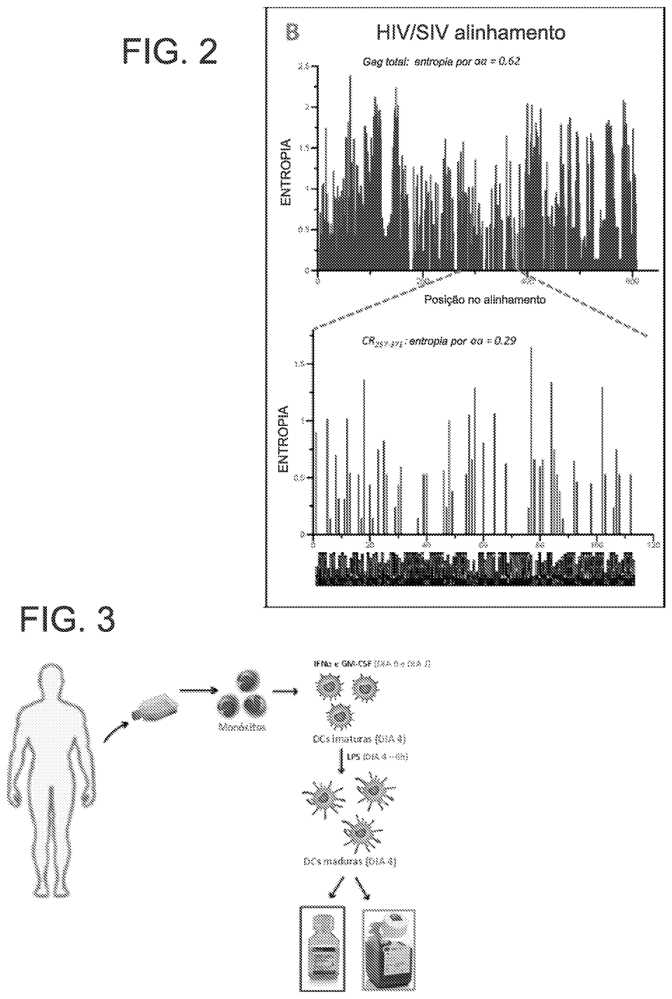

The inventor, through this document, teaches a novel approach for the development of a personalized vaccine. This approach is based on: A) sequencing of the gag gene from an HIV- infected individual treated with antiretroviral therapy; B) sequencing of the HLA alleles of the same individual; C) selecting the epitopes recognized by the individual's own HLA Class I within the highly-conserved Gag₂₅₆₋₃₇₇, Gag₁₄₇₋₁₆₉ and/or Gag₂₂₅₋₂₅₁ amino acid sequences (Los Alamos HIV Database), shown in FIG. 2. Preference is given to peptides with good binding strength and high affinity for the individual's Class I HLA and to sequences showing a high binding affinity for the same individual's HLA Class II. Small 9-mers may maximize HLA Class I presentation and the immune response thereupon.

For the purposes of clarification, in bioinformatics, k-mers are subsequences of k length contained in a biological sequence. They are mainly used in the context of computational genomics and sequence analysis, in which k-mers are composed of amino acids that form protein sequences with the function of improving the expression of the heterologous gene, also identifying species in metagenomics samples, potentially creating attenuated vaccines.

An original algorithm that designs the target peptide for the vaccine starting from viral and HLA sequences of an individual with HIV/AIDS, forms the core of the present invention (see Example 1). The original algorithm makes extensive use of existing open-source software for protein design. The peptides designed in this manner and accordingly synthesized may be exploited as a therapeutic vaccine against HIV/AIDS. Vehicles for such peptides may be an individual's own dendritic cells pulsed with the peptide combination (FIG. 3) or a specific viral or DNA vector leading to intracellular expression of the viral peptides. The present vaccine approach may contribute to control of viremia once antiretroviral therapies are suspended (see Example 1).

Explained so far in a summarized form, the invention is now better detailed through the attached figures:

FIG. 1—Left. Capsid protein structure showing hexamers of hexamers of the lentiviral capsid protein. Right: structural insights on the viral capsid protein. Panels A, B: ribbon representation of the protein showing (A) the N-terminal (dark green) and the C-terminal (light green) domains and (B) the highly conserved regions (yellow). Panels C,D: Three-dimensional representation of a hexamer: WROM visualization from the outside (C) and from within (D). The highly-conserved region is mapped in the same color (yellow) on the protein surface.

FIG. 2—Shannon entropy (the higher the score, the higher the sequence variability) for an HIV/SIV alignment and for the highly conserved region at the C-terminal of HIV-1 p24 (from: Shytaj e Savarino , J Med Primatol 2015);

FIG. 3—Schematic view of the maturation process of dendritic cells deriving from monocytes using IFNα as part of the initial phase of the interleukin cocktail;

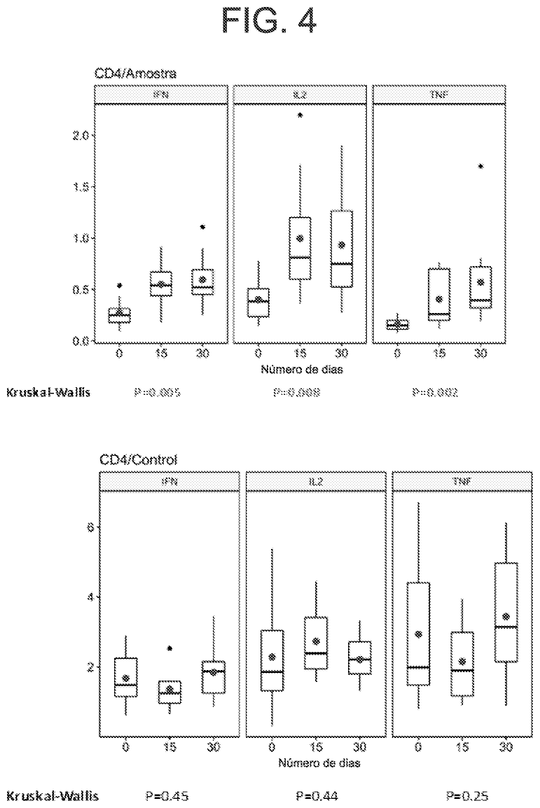

FIG. 4—Ex-vivo immunogenicity analysis of the vaccine proposed here in CD4+ T cells, where the X axis shows the time points and the y axis the percentage of cytokine producing cells after stimulation. Asterisks show significant differences from baseline. The panel above shows the stimulation of PBMCs (peripheral blood mononuclear cells) of patients with the peptides adopted for immunization. The panel below shows the stimulation of cells with non-vaccine related stimuli (see Example 1);

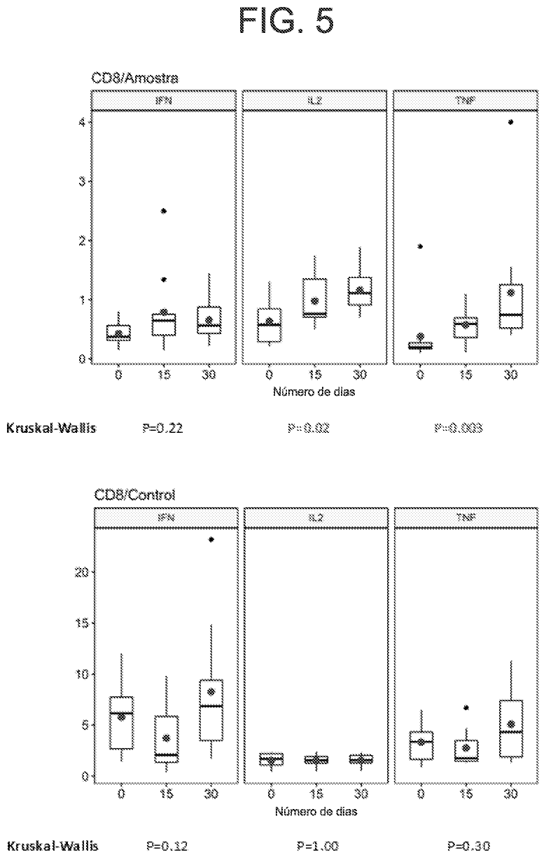

FIG. 5—Ex vivo immunogenicity analysis of the vaccine proposed here in CD8+ T cells. The X axis shows the time points and the y axis shows the percentage of cytokine producing cells after stimulation. Asterisks show significant differences from baseline. The panel above shows the stimulation of PBMCs of patients with the peptides adopted for immunization. The panel below shows the stimulation of cells with non-vaccine related stimuli (see Example 2);

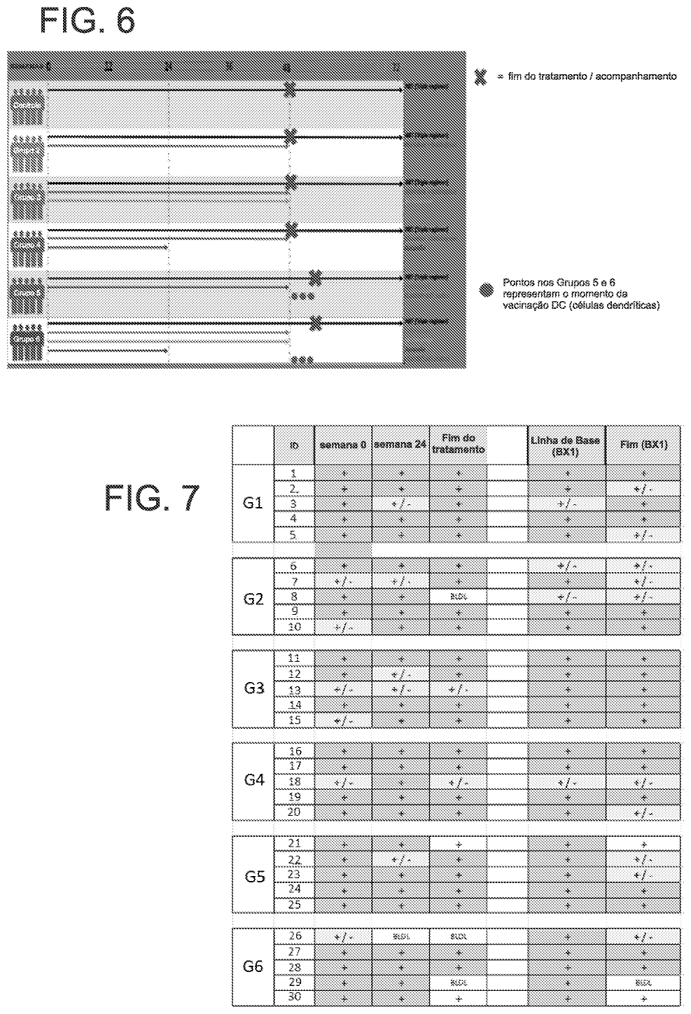

FIG. 6—Schematic representation of treatments administered to HIV+ individuals;

FIG. 7—Results of viral DNA quantification in subjects that received the personalized vaccine herein disclosed following an experimental treatment consisting of auranofin, nicotinamide and intensified antiretroviral therapy. * Black asterisks below the graph represent the significant pre- and post-treatment differences in individual subjects according to post-hoc analysis. PBMC: peripheral blood mononuclear cells. RB: rectal biopsies;

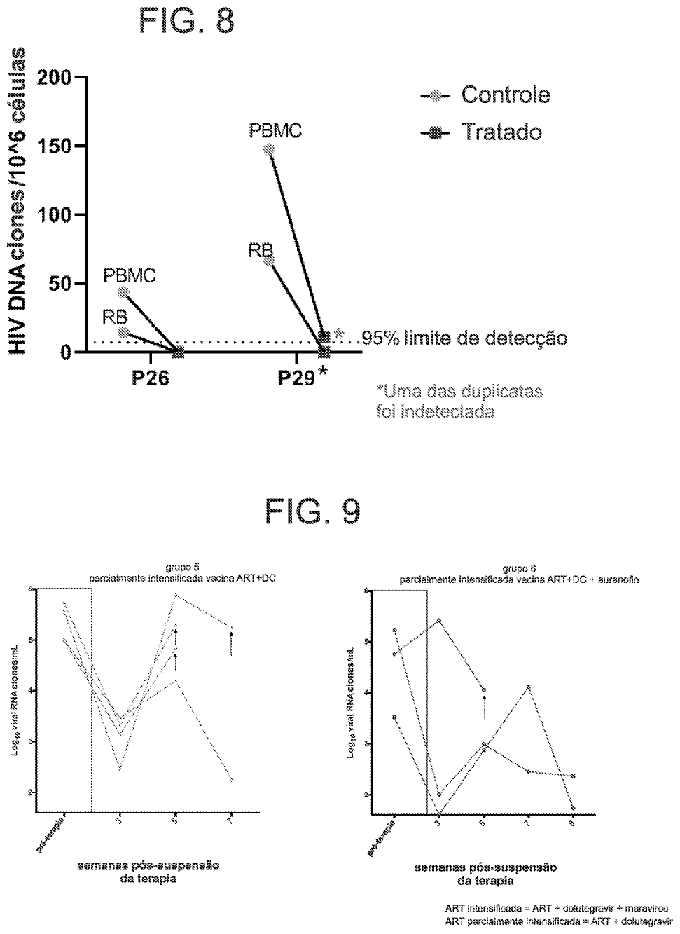

FIG. 8—Post-therapy viral loads in patients treated with the experimental vaccine.

Arrows indicate those patients for whom the peptides were designed according to a different protocol from that disclosed and claimed in the present invention and who resumed antiretroviral therapy due to viral rebound to unacceptable levels

### DETAILED DESCRIPTION OF THE INVENTION

In accordance with the attached drawings, the “METHOD FOR DEFINING A PERSONALIZED VACCINE AGAINST HIV/AIDS”, object of this patent application, is a novel and never before attempted approach to design a vaccine against HIV/AIDS (or Human Immunodeficiency Virus—HIV). The present invention is based on a personalized medicine approach, which distinguishes it from approaches attempted so far that are limited to exploiting highly conserved gag regions in a general vaccine.

In the first step, HIV-1 gag DNA sequences derived from DNA extracted from a patient's peripheral blood mononuclear cells (PBMCs) are translated to amino acids in the correct reading frame, as shown in Example 1, shown further in this document. Human Leukocyte Antigen (HLA) haplotypes are sequenced in parallel.

In the second step, amino acid sequences are aligned and a consensus sequence is created. Additional alignments are then compared with published sequence alignments to map the highly-conserved regions to the individual's viral gag consensus sequence. Possible errors and/or uncertain positions are then corrected either manually based on sequence alignments, or automatically, as shown in Example 1.

In the third step, the epitopes to be adopted in the vaccine are chosen among those that are best recognized by the patient's HLA Class I according to automated calculations based on the above consensus sequences. The criteria for determination of a peptide were its ability to dock to HLA-I and HLA-II binding sites as indicated by the IEDB (Immune Epitope Database and Analysis Resource). Their positions are then validated based on biological data on peptides in the corresponding regions, as reported by the Los Alamos HIV database (https://www.hiv.lanl.gov/content/immunolog/maps/cd/Gag.html). Only peptides showing high binding affinity (>100; IEDB score) and residing in protein positions documented to interact with the individual's HLA Class I are selected to be included in the vaccine preparation. In case of low binding affinities (IEDB score <100) of epitopes within the Gag₂₅₆₋₃₇₇ sequence or in case of lack of documented interactions of the considered HLA haplotype with peptides in corresponding positions, the Gag₁₄₇₋₁₆₉ and Gag₂₂₅₋₂₅₁ sequences (FIG. 2) will be explored and immunogenic peptides will be selected therefrom (Table 1). The same task should be performed in case there are less than two peptides deriving from Gag₂₅₆₋₃₇₇ and adherent to these criteria. Preferably, high affinity HLA Class I binding peptides should be selected from sequences also showing good binding affinity with HLA Class II, although our immunogenic peptide design is not limited by this process.

HLA works by bringing viral fragment peptides to the surface of an infected cell where the host's immune system can recognize and kill them. These fragments are generally 9 amino acids in length. For this reason and other reasons related to fabrication of the personalized peptide, the final stage of the peptide design is to reduce the size of the designed peptide to 9-mers. The manufacturing constraints relate to the possibility of creating peptides with loops that would make parts of the peptide unavailable to the host's immune system or that could create electrical interactions among amino acids that also would effectively seal off parts of the peptide to perception by the host. For this reason, the peptides must be evaluated using the ProtParam database on the ExPASy server, to test whether the peptide will be sufficiently persistent and open to allow binding to dendritic cells.

Peptides derived from this process are synthesized and purified according to Example 2 (described further below) and used to pulse dendritic cells from the same HIV-infected subject (FIG. 3).

A variation of this invention may result in a preventive vaccine against HIV, by following the same method as above but using, instead of an individual's own viral sequences, consensus sequences or a mosaic of sequences of Gag₂₅₆₋₃₇₇ heterologous viruses (from other individuals) from epidemiologically relevant viruses within the region where the individual resides.

### EXAMPLE 1

Algorithm for the workflow adopted in the present invention:
1. Translate DNA into amino acid sequence
   Align the three types of translation to the start of gag in the HXB-2 reference sequence to determine the correct reading frame (the usual decision is to choose the frame which produces fewest indeterminate results);
   a) Software commonly used: Clustal Omega or Bioconductor Biostrings (although there are many alternatives).
2. Edit resultant polypeptide by manual correction, i.e. replace tryptophans by indications of start codon in the middle of sequence.
3. Determine HLA haplotypes for Locus A, B and C and HLA II from sequencing.
4. Test the fitness of the amino acid HIV gag sequences sequence for the HLA I subtype from the same patient
   a) Using the HLA Peptide Binding Predictions page at www-bimas.cit.nih.gov/molbio/hla_bind/:
   b) In the case of patient LMC, this would be A1 and A33 (see Table 1);
   c) The software produces a set of 9-mers scored from highest to lowest;
   d) Search for high scoring regions that are repeated across multiple types of HLA molecules (groups);
   e) Selection of peptides that reside in the highly conserved region (codons 257-371 in the codon 431 sequence we are using).
5. Repeat process for Type II Binding regions
   a) a. Using the IEDB Analysis Resource (http://tools.iedb.org/mhciik
   b) Select, wherever possible, those peptides from high-scoring regions also for HLA Class I binding.
6. Determine by examination a 9-mer to a 30-mer that incorporates the maximum number of the high-scoring haplotypes.
7. Test the parameters of the resulting peptide against the ProtParam program at the Expasy website (https://web.expasy.org/cgi-bin/protoparam/protoparam) to determine its composition, estimated half-life and stability.

### EXAMPLE 2

### Personalized Determination of the Peptides used for Each Patient

Given the impossibility of autologous control of HIV-1 in people on long-term suppressive antiretroviral therapy, we designed a personalized vaccine with dendritic cells for each of the study subjects in a clinical trial. The HLA profile determined for the individuals in the study can be found in Table 1 below:

TABLE 1
HLA profile determined for each of the volunteers in Groups 5 and 6, including the
steps needed to manufacture a vaccine with dendritic cells. ID: candidate identity.

| ID | Locus A* | | Locus B* | | Locus C* | | Locus DRB1* | |
|---|---|---|---|---|---|---|---|---|
| 25 | 02: AJEBB | 03: AJEYV | 18: AEDBZ | 40: AEDCG | 03: AJFXY | 05: AJFZD | 07: ANCXR | 13: AKKUB |
| 22 | 01: AJEVP | 03: AJEZG | 35: AGNUR | 44: AGKXV | 04: AJTUU | 16: AJSFG | 04: VVSK | 08: AKKFM |
| 23 | 01: AJHDX | 33: AJHHP | 37: AGKTW | 58: AGMCF | 03: AJSBD | 06: AJWYX | 04: AEEWN | 07: ANBTH |
| 21 | 01: AJEVS | 24: AJFBS | 40: AGHBM | 51: AGHCW | 02: AJRUK | 15: AJRUU | 04: VVSK | 13: AGKBM |
| 24 | 02: AJEXK | 24: AJFBM | 14: AGRAG | 51: ZUDX | 02: AJRUK | 15: AJRUU | 01: ADXMM | 08: AGFNT |
| 30 | 02: AJSFV | 02: AEDPZ | 15: AGRAS | 57: BNK | 02: AJGWH | 03: AJGWT | 04: ANEGN | 16: ANBUK |
| 29 | 02: AJEAZ | 02: AJEAZ | 15: AGKPJ | 51: AGKZJ | 04: AJFYM | 14: AGTAE | 11: ANTEX | 11: AGMJS |
| 27 | 23: AHPYX | 26: AJFCW | 14: ADJFT | 44: AGRGS | 04: AJKCT | 08: AJKEG | 07: ANCXN | 07: ANCXN |
| 26 | 02: AJEBB | 03: AJEYV | 07: AGKMM | 13: AGKNX | 06: AJKDJ | 07: AJKEA | 03: ANCWU | 10: VDG |
| 28 | 02: AJEAZ | 02: AJEAZ | 07: AGKMF | 51: AGMAB | 07: AJHZC | 16: AJJAB | 15: ANBUC | 16: ANBUG |

The first step was the determination of the DNA sequences of the HIV-1 gag gene region. We extracted the DNA from PBMCs of each patient. At least 10 clones were determined per patient by the technique known as “single genome amplification” or “endpoint PCR” [Diaz et al., 1997].

In the second step, when the HIV DNA sequences were translated to amino acid sequences, they were aligned using Clustal-Omega, and a consensus sequence was created, although each separate sequence for the same patient was retained for the possible creation of a peptide with high antigenic power. Additional alignments were then made using the published aligned sequences with the aim of mapping the highly conserved regions of the consensus gag sequences for each of the study subjects. Incorrect positions and other errors were manually corrected based on sequence alignment. The epitopes to be targeted in the vaccine were derived from those calculated to be best recognized by the HLA Class I of the same patient and double-checked against sequences validated by the Los Alamos National Laboratory's HIV database (https:/www.hiv.lanl.gov/content/immunology/maps/ctl/Gag.html), as described in the main text.

Only those peptides that showed a high binding affinity (IEDB score >100) and mapping to positions on the protein previously documented to interact with the subject's HLA Class I were selected as vaccine candidates. Several peptides were selected in positions covered by codons 256-377 of the gag region. As such, we designed 2 to 6 peptides per candidate, as shown below in Table 2:

TABLE 2
Description of peptides designed for each patient
(9-mers) to achieve the best immunogenicity as
described above. Note that some autologous
peptides are common to more than one patient.

| ID/Grupo | PEPTÍDEO |
|---|---|
| 24 | 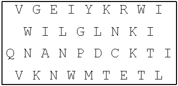 |
| 25 | W I I L G L N K I |
| | Y V D R F Y K T L |
| | K A L G P A A T L |
| 23 | P E V I P M F S A |
| | F S P E V I P M F |
| 22 | V H E K K E V R D |
| | K E V R D T K E A |
| | T I K C F N C G K |
| | G P K R T I K C F |
| | T L Y C V H E K K |
| | C V H E K K E V R |
| 21 | W I I L G L N K I |
| | G L N K I V R M Y |
| | F R D Y V D R F Y |
| | R A E Q A S Q E V |
| 29 | 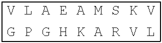 |
| 28 | W I I L G L N K I |
| | Y V D R F Y K T L |
| | K A L G P A A T L |
| 30 | W I I L G L N K I |
| | Y V D R F Y K T L |
| | K A L G P A A T L |
| 26 | 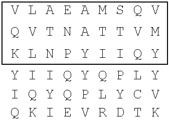 |
| 27 | K V K N M T E S L |
| | L R L N K I V R M |
| | A E W D R L H P V |

### Peptide Synthesis

An automatic desktop synthesizer (Shimadzu PSSM 8) was used for the simultaneous solid phase synthesis of all peptides using the Fmoc procedure. The final peptides were “unprotected” in TFA and purified by HPLC semi-preparation using an Econosil C-18 column (10 μ, 22.5×250 mm) and a two-solvent system: (A) trifloroacetic acid (TFA)/H₂O (1:1000) and (B) TFA/acetonitrile (ACN)/H₂O (1:900:100). The column was eluted at a flow rate of 8 mL/min with a gradient of 0 to 80% solvent B over 45 minutes. The HPLC analysis was done using a binary HPLC system manufactured by Shimadzu with a UV-vis detector SPD-10AV (Shimadzu), coupled to an Ultrasphere C-18 column (5 μ, 4.6×150 mm) that was eluted with system solvents A1 (TFA/ H₂O, 1:1000) and B1 (ACN/H₂O/TFA, 900:100:1) at a flow rate of 1.0 mL/min and a gradient of 10-80% of B1 over 10 minutes. The eluates from the HPLC columns were monitored for their absorbance at 220 nm. The molecular weight and the purity of the synthesized proteins were verified by electron spray (LC/MS-2010 Shimadzu). The quantity of peptide was determined by analysis of the aminoacids (Shimadzu).

### Cytapheresis of the Patients for the Manufacturing of the Dendritic Cell Vaccine

Autologous mononuclear cells were collected from the participants and underwent leukapheresis using the Terumo Cobe Spectra cellular separator at the Sao Paulo Blood Center (Hospital das Clínicas).

For each participant, the total blood volume was calculated and 1.5 times this volume was processed in a continuous flow at a rate of 50-60 mL/min using peripheral venal access.

After blood collection, the product was sent to the Retrovirology Laboratory of UNIFESP for purification of monocytes and subsequent transformation into dendritic cells. During these procedures only minor adverse events were noted such as perineural paresthesia in the fingertips.

### Dendritic Cell Vaccine Preparation for Administration to HIV-Infected Individuals

The protocol details are as follows: the apheresis product (approximately 130 mL) was diluted 1:2 in saline solution (0.9% NaCl) and separated by a density gradient using Ficoll®-Paque Premium (GE Healthcare®). After centrifugation at 800 g for 30 minutes at a temperature of 15° C., the cloud of peripheral blood mononuclear cells (PBMC) was removed and subjected to two rinses at 600 g for 10 minutes at 15° C.

The PBMCs obtained were quantified and evaluated by optical microscope for the calculation of cellular viability in a Neubauer chamber utilizing a Trypan 0.4% (Sigma-Aldrich®) blue dye. Aliquots containing 5×10⁷ cells/mL were cryopreserved in a medium of 10% de Dimethyl sulfoxide (DMSO-Sigma®) in certified fetal bovine (SFB—Gibco Life Technologies®) for differentiation between monocytes and dendritic cells. The cells were stored in liquid nitrogen until their use.

On day 0, for the differentiation of dendritic cells, aliquots of PBMCs that were previously obtained by apheresis were thawed in a water bath at 37° C. After two rinses with saline solution for 10 minutes at 15° C., the material was quantified and its viability evaluated. Following this, aliquots of 5×10⁶ cells/mL were added to the culture medium RPMI 1640 (Gibco®) and the cells were conditioned in 25 cm² culture flasks and incubated in CO₂ at 37° C. for 1.5 hours so that the monocytes might separate by adhering to the plastic.

After this time, non-adherent cells (predominantly T lymphocytes, B lymphocytes and NK cells) were removed by rinsing. Adherent cells (predominantly monocytes) were maintained in culture medium AIM-V (Gibco®) and 100 ng/mL of GM-CSF and 500 IU/mL of IFN-α2b were added. After a 24-hour culture, the same quantities of cytokines GM-CSF and IFN-α2b were added. On day 2, the HIV peptides were added (0.2 μg/mL de each peptide) and incubated overnight. On day 3 of the culture, 6 hours before extracting the cells for the activation of the dendritic cells, 5 EU/mL of LPS were added to the culture flasks. After the incubation, the DCs were recovered with the help of an ice bath and were rinsed three times with saline solution.

The study subjects received the DC vaccine in accordance to the protocol, after Week 48 of the study. They received 3 doses of the vaccine with an interval of 15 days between them. To assess the immunogenicity of the vaccine, new samples were collected immediately before the first dose (baseline), immediately before the second dose (reflecting the immunogenic effect of the first dose) and immediately before the third dose (reflecting the impact of the second dose). At this time, we also obtained rectal biopsies for patients in the two groups. Evaluation of immunogenicity in CD4+ and CD8+ T cells by quantifying IL-2, TNF and INF by flow cytometry.

It is worth noting that before the administration of each dose of vaccine, cell viability and the phenotypic profile of DCs present in the dose were evaluated and optimized (data not shown). This was important as the second and third doses were prepared from frozen PBMCs.

For the in vitro analysis of the dendritic cell vaccine that stimulated with the autologous HIV peptide, two heparin-containing tubes were collected from each patient before inoculation with each of the three doses of the vaccine. Peripheral blood mononuclear cells (PBMCs) from each patient were gradient separated with Ficoll®-Paque. One million cells per well were placed in RPMI culture medium with 10% fetal bovine serum and the autologous peptides were added at a concentration of 1 μg/mL. One well in the plate was kept as a control sample. The peptide was not added to this well. The 96-well plate (with a u-shaped bottom) was placed in a CO₂ incubator at 37° C. for 48 hours. During the last 6 hours, the positive control received enterotoxin from S.aureus type B (SEB) and Brefeldin A (BFA). Cells were analyzed in an intracellular flow cytometer with quantification of IL2, TNF and IFN in CD4+ and CD8+ T cells with correct comparisons between the immunogenicity of samples and controls. Note that the results at the first time point, which relate to each candidate receiving their individual vaccine, reflect the baseline cellular response status of the autologous HIV peptides, while the results at the second time point reflect the immunogenic impact of the first dose of the vaccine. Likewise, the third time point, that is, the time of administration of the third dose of the vaccine, reflects the immunogenic impact of the second dose of the vaccine. At timepoints 2 and 3, the number of interleukin-producing cells significantly increased in CD4+ and CD8+ T cells, providing proof of concept for the immunogenicity of this vaccine approach.

Finally, table 3 below shows the qualitative results of total HIV DNA in PBMCs (left) and rectal biopsy tissues (BX1; right) over time. In yellow, patients who deviated from the protocol by interrupting therapy on their own initiative are shown:

1. A method for defining a personalized vaccine against HIV/AIDS consisting of a combination of antigenic peptides from the gag gene of the virus, said method comprising the following steps:
a) sequencing of the HIV-1 gag gene of the circulating viral quasi-species in peripheral blood mononuclear cells of an antiretroviral-treated HIV positive individual displaying indetectable viral load;
b) sequencing of the HLA locus of the same patient, so as to determine the HLA haplotypes;
c) translation into amino acid sequences of the circulating HIV gag DNA sequences;
d) sequence alignment and creation of a consensus sequence of the patient's Gag proteins;
e) in-silico calculation of the best epitopes (9-mers) within the highly-conserved sequences (from the previous step) capable of interacting with the patient's HLA Class I haplotypes;
f) selection of epitopes (2 to 6 peptides among the best epitopes resulting from the in silico calculation) corresponding to a standard HIV proteins sequence, where an interaction with the Class I HLA haplotypes in question was mapped from previous biological data, where the criteria for determination of a peptide were its ability to dock to HLA-I and HLA-II binding sites as indicated by the IEDB (Immune Epitope Database and Analysis Resource), positions then validated against biological data from peptides in the corresponding regions, as reported by the Los Alamos HIV database, and where only peptides showing high binding affinity (>100; IEDB score) and residing in protein positions documented to interact with the individual's HLA Class I are selected;
g) preferential selection, whenever possible, of epitopes that correspond to amino acid positions that are not variable in the viral quasi-species sequenced from the patient's peripheral blood mononuclear cells;
h) selection of those epitopes less likely to form loops (which would make parts of the peptide unavailable to the host's immune system or that could create electrical interactions among amino acids), and being most stable according to isolated calculations;
i) synthesis of the peptides;
j) ex-vivo pulsing with the peptide combination of the patient's own dendritic cells.

2. A method for defining a personalized vaccine against HIV/AIDS according to claim 1, using a combination of peptides (minimum of two, maximum of seven) based on the following order of preference based on position within the gag sequence: Gag₂₅₆₋₃₇₇>Gag₁₄₇₋₁₆₉≥Gag₂₂₅₋₂₅₁.

3. A method for defining a personalized vaccine against HIV/AIDS according to claim 1, wherein the epitopes are preferentially selected from those repeatedly top ranking for more than one of the patient's HLA Class I haplotypes.

4. A method for defining a personalized vaccine against HIV/AIDS according to according to claim 1, wherein the vaccine is administered after a course of treatment with one or more antiproliferative agents.

5. A method for defining a personalized vaccine against HIV/AIDS according to claim 4, wherein antiproliferative agents are administered during antiretroviral therapy.

6. A method for defining a personalized vaccine against HIV/AIDS according to according to claim 4, wherein the antiproliferative agents are auranofin and/or nicotinamide.

7. A method for defining a personalized vaccine against HIV/AIDS according to claim 4, wherein the administration of auranofin is associated with an agent that intensifies its antiproliferative effect.

8. A method for defining a personalized vaccine against HIV/AIDS according to according to claim 5, wherein the antiproliferative agents are auranofin and/or nicotinamide.

9. A method for defining a personalized vaccine against HIV/AIDS according to claim 5, wherein the administration of auranofin is associated with an agent that intensifies its antiproliferative effect.

10. A method for defining a personalized vaccine against HIV/AIDS according to according to claim 6, wherein the administration of auranofin is associated with an agent that intensifies its antiproliferative effect.

11. A method for defining a personalized vaccine against HIV/AIDS according to claim 2, wherein the epitopes are preferentially selected from those repeatedly top ranking for more than one of the patient's HLA Class I haplotypes.

12. A method for defining a personalized vaccine against HIV/AIDS according to according to claim 2, wherein the vaccine is administered after a course of treatment with one or more antiproliferative agents.

13. A method for defining a personalized vaccine against HIV/AIDS according to claim 12, wherein antiproliferative agents are administered during antiretroviral therapy.

14. A method for defining a personalized vaccine against HIV/AIDS according to according to claim 13, wherein the antiproliferative agents are auranofin and/or nicotinamide.

15. A method for defining a personalized vaccine against HIV/AIDS according to claim 13, wherein the administration of auranofin is associated with an agent that intensifies its antiproliferative effect.

16. A method for defining a personalized vaccine against HIV/AIDS according to according to claim 3, wherein the vaccine is administered after a course of treatment with one or more antiproliferative agents.

17. A method for defining a personalized vaccine against HIV/AIDS according to claim 16, wherein antiproliferative agents are administered during antiretroviral therapy.

18. A method for defining a personalized vaccine against HIV/AIDS according to according to claim 17, wherein the antiproliferative agents are auranofin and/or nicotinamide.

19. A method for defining a personalized vaccine against HIV/AIDS according to claim 17, wherein the administration of auranofin is associated with an agent that intensifies its antiproliferative effect.
